(19) United States
(12) Patent Application Publication
(10) Pub. No.: US 2025/0283805 A1
(43) Pub. Date: Sep. 11, 2025

(54) URINE ANALYSIS DEVICE

(71) Applicant: DNC BIOTECHNOLOGY INC., Daejeon (KR)

(72) Inventor: Seong Su PARK, Anseong-si (KR)

(21) Appl. No.: 19/220,240

(22) Filed: May 28, 2025

Related U.S. Application Data
(63) Continuation of application No. PCT/KR2023/019251, filed on Nov. 27, 2023.

(30) Foreign Application Priority Data
Aug. 29, 2023 (KR) 10-2023-0113746

(51) Int. Cl. G01N 21/29; B01L 3/00; G01N 33/543

(52) U.S. Cl. CPC G01N 21/29; B01L 3/5023; G01N 33/54391; B01L 2200/02; B01L 2300/0825; B01L 2400/0406

(57) ABSTRACT

A urine analysis device according to an embodiment of the present disclosure includes: a mounting unit in which a test kit is mounted, the test kit including a container into which urine is collected and a curved test strip which is attached along an inner or outer circumferential surface of the container and includes a plurality of test papers provided to test the collected urine; a plurality of sensor units, each composed of a pair of a light emitting unit configured to emit light onto each of the plurality of test papers of the test kit mounted on the mounting unit and a light receiving unit configured to detect a color of each test paper based on the light reflected from each of the plurality of test papers; and an analysis unit that analyzes the urine based on the color information of the test papers detected by the light receiving units.

### TECHNICAL FIELD

The present disclosure relates to a urine analysis device.

### BACKGROUND

In general, a urine test is an essential examination that occupies about 30% of the total number of examinations performed in most hospitals or clinics. The urine test is a method to examine the components of urine to assess systemic conditions and detect lesions.

A conventional urine test is performed to examine urine by bringing a test stick with a paper test strip into contact with an examinee's urine and checking a color change of the paper test strip. That is, an examiner performs the urine test by directly applying the examinee's urine to the test stick, or by collecting urine in a container, such as a paper cup, and bringing the test stick into contact with the collected urine.

When a urine test is performed by using a urine analysis device including a urine test kit equipped with a flat test strip, the test is performed by emitting light over the entire area of the flat test strip and receiving the reflected light to determine the color. However, when the test is performed by using a urine test kit equipped with a curved test strip, it is difficult to determine the color of the entire area of the curved test strip by the conventional test method.

Also, if urine test kits are manufactured with varying degrees of curvature, such as differences in the radius of curvature or radius, urine analysis devices capable of accurate detection are required for each type.

### DISCLOSURE OF THE INVENTION

### Problems to be Solved by the Invention

The present disclosure is conceived to provide a device for analyzing urine by using a urine test kit equipped with a curved test strip.

However, the problems to be solved by the present disclosure are not limited to the above-described problems. There may be other problems to be solved by the present disclosure.

### Means for Solving the Problems

According to an aspect of the present disclosure, a urine analysis device includes: a mounting unit in which a test kit is mounted, the test kit including a container into which urine is collected and a curved test strip which is attached along an inner or outer circumferential surface of the container and includes a plurality of test papers provided to test the collected urine; a plurality of sensor units, each composed of a pair of a light emitting unit configured to emit light onto each of the plurality of test papers of the test kit mounted on the mounting unit and a light receiving unit configured to detect a color of each test paper based on the light reflected from each of the plurality of test papers; and an analysis unit that analyzes the urine based on the color information of the test papers detected by the light receiving units.

According to another aspect of the present disclosure, a urine analysis device includes: a mounting unit in which a test kit is mounted, the test kit including a container into which urine is collected and a curved test strip which is attached along an inner or outer circumferential surface of the container and includes a plurality of test papers provided to test the collected urine; a substrate unit provided on an inner surface of the mounting unit and formed of a ductile material; a sensor unit provided on the substrate unit and composed of a light emitting unit configured to emit light onto the plurality of test papers and a light receiving unit configured to detect a color of the plurality of test papers based on the light reflected from the plurality of test papers; and an analysis unit that analyzes the urine based on the color information of the test papers detected by the light receiving unit.

The above-described aspects are provided by way of illustration only and should not be construed as liming the present disclosure. Besides the above-described embodiments, there may be additional embodiments described in the accompanying drawings and the detailed description.

### Effects of the Invention

According to any one of the above-described means for solving the problems of the present disclosure, it is possible to more accurately perform a urine test even when a urine test kit equipped with a curved test strip is used.

### BRIEF DESCRIPTION OF THE DRAWINGS

FIG. 1 is a schematic diagram illustrating a urine analysis device according to an embodiment of the present disclosure.

FIG. 2 is a configuration view of the urine analysis device according to an embodiment of the present disclosure.

FIG. 3 shows a urine test kit mounted on the urine analysis device according to an embodiment of the present disclosure.

FIG. 4 is a planar figure of a substrate unit of the urine analysis device according to an embodiment of the present disclosure.

FIG. 5 illustrates the state in which the substrate unit shown in FIG. 4 is disposed to surround the urine test kit.

FIG. 6A is a diagram for explaining a method of acquiring color information of each of a plurality of test papers by emitting light from the urine analysis device according to an embodiment of the present disclosure.

FIG. 6B is a diagram for explaining a method of acquiring color information of each of a plurality of test papers by emitting light from the urine analysis device according to an embodiment of the present disclosure.

FIG. 7 is a flowchart of a urine analysis method using the urine test kit according to an embodiment of the present disclosure.

### BEST MODE FOR CARRYING OUT THE INVENTION

Hereinafter, embodiments will be described in detail with reference to the accompanying drawings so that the present disclosure may be readily implemented by a person with ordinary skill in the art. However, it is to be noted that the present disclosure is not limited to the embodiments but can be embodied in various other ways. In the drawings, parts irrelevant to the description are omitted for the simplicity of explanation, and like reference numerals denote like parts throughout the whole document.

Throughout the whole document, the term “connected to” or “coupled to” that is used to designate a connection or coupling of one element to another element includes both a case that an element is “directly connected or coupled to” another element and a case that an element is “electronically connected or coupled to” another element via still another element. Further, it is to be understood that the term “comprises or includes” and/or “comprising or including” used in the document means that one or more other components, steps, operation and/or existence or addition of elements are not excluded in addition to the described components, steps, operation and/or elements unless context dictates otherwise and is not intended to preclude the possibility that one or more other features, numbers, steps, operations, components, parts, or combinations thereof may exist or may be added.

Throughout the whole document, the term “unit” includes a unit implemented by hardware or software and a unit implemented by both of them. One unit may be implemented by two or more pieces of hardware, and two or more units may be implemented by one piece of hardware.

In the present specification, some of operations or functions described as being performed by a device may be performed by a server connected to the device. Likewise, some of operations or functions described as being performed by a server may be performed by a device connected to the server.

Hereinafter, embodiments of the present disclosure will be described in detail with reference to the accompanying drawings.

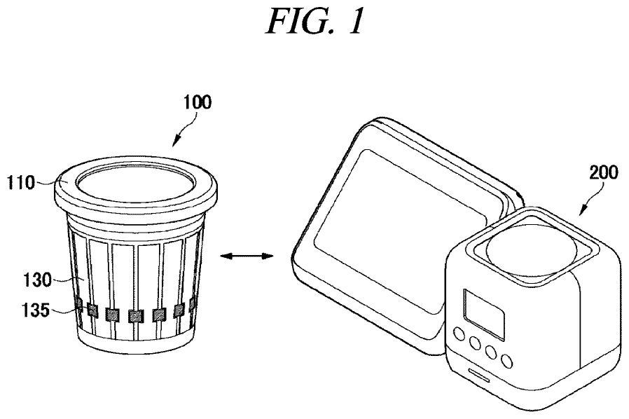

FIG. 1 is a schematic diagram illustrating a urine analysis device according to an embodiment of the present disclosure.

Referring to FIG. 1, a urine analysis device 200 according to an embodiment of the present disclosure is configured to analyze urine collected into a cup-type urine test kit 100 from an examinee by detecting the color of a test paper 135 of a curved test strip 130 which reacts with the urine of the examinee.

In an embodiment of the present disclosure, the urine test kit 100 may include a container 110 and the curved test strip 130 attached along an inner or outer circumferential surface of the container 110.

Specifically, as shown in FIG. 1, the urine test kit 100 may include the container 110 into which urine is collected, and the curved test strip 130 which is attached along the inner or outer circumferential surface of the container 110 and includes a test paper 135 provided to test the urine collected in the container 110.

For example, the curved test strip 130 may be attached along the inner or outer circumferential surface of the container 110 with an appropriate curvature, depending on the size of the container 110 and the length of the curved test strip 130.

The curved test strip 130 may individually accommodate test papers 135, which react with urine, in separate compartments and may include flow paths that allow urine to flow into the respective compartments.

In this case, a plurality of test papers 135 may be formed corresponding in number to various test items. For example, the test papers 135 may be 11, 7, or 4 in number, as shown in Table 1.

TABLE 1

| | | Initial | 11-Point | 7-Point | 4-Point |
|---|---|---|---|---|---|
| No. | Test Item | Color | Kit | Kit | Kit |
| 1 | Occult Blood | Color a | ∘ | ∘ | ∘ |
| 2 | Bilirubin | Color b | ∘ | — | — |
| 3 | Urobilinogen | Color c | ∘ | ∘ | — |
| 4 | Ketones | Color d | ∘ | ∘ | — |
| 5 | Protein | Color e | ∘ | — | ∘ |
| 6 | Nitrite | Color f | ∘ | ∘ | — |
| 7 | Glucose | Color g | ∘ | ∘ | ∘ |
| 8 | pH | Color h | ∘ | ∘ | — |
| 9 | Specific Gravity | Color i | ∘ | — | — |
| 10 | Leukocytes | Color j | ∘ | ∘ | ∘ |
| 11 | Vitamin C | Color k | ∘ | — | — |

Referring to Table 1, the test items may include occult blood (OBD), bilirubin (BIL), urobilinogen (URO), ketones (KET), protein (PRO), nitrite (NIT), glucose (GLU), pH, specific gravity (SG), leukocytes (LEU), and vitamin C. Urine analysis can be performed by using the urine test kit 100 which includes 11, 7, or 4 test papers 135 depending on the purpose of the test.

The flow path may be divided into a lower flow path connected to a lower side of the test paper 135 and an upper flow path connected to an upper side of the test paper 135. When an air pump (not shown) attached to the upper flow path is pressed, urine collected in the container 110 flows through the lower flow path and wets the test paper 135.

The container 110 may be cylindrical or cup-shaped and formed of a transparent plastic material to facilitate urine collection from the examinee and to allow visual inspection of the collected urine.

In an embodiment, when the container 110 is mounted in a mounting unit of the urine analysis device 200, which will be described later, a protrusion or groove for guiding the container 110 to a correct mounting position may be formed at the bottom of the container 110. Conversely, a groove or protrusion corresponding to the protrusion or groove of the container 110 may be formed in the mounting unit of the urine analysis device 200.

Meanwhile, a tag or code for identifying the type of the urine test kit 100 and recording information of the examinee may be attached to one surface of the container 110.

For example, an NFC reader (not shown) may be attached to the one surface of the container 110. The NFC reader is configured to read recorded data from an NFC tag attached to the urine test kit 100. The NFC tag may record information, such as the examinee's data and the type of urine test kit 100 indicating whether it is a 11-point kit, a 7-point kit, or a 4-point kit. Although the NFC tag has been described as an example in the embodiment of the present disclosure, RFID, barcodes, or QR codes may also be used.

The urine analysis device 200 according to an embodiment of the present disclosure may be a device that analyzes urine by using the urine test kit 100 equipped with the curved test strip 130.

The urine analysis device 200 of the present embodiment may be configured to allow the urine test kit 100 to be mounted. Once the urine test kit 100 is mounted, the urine analysis device 200 emits light onto the test papers 135 of the curved test strip 130, receives light reflected from the test papers 135, and analyzes the urine based on color information of the reflected light. The urine analysis device 200 may include a display to present the analysis result of the urine.

The urine analysis device 200 according to the present embodiment can accurately perform a urine test even when the urine test kit 100 equipped with the curved test strip 130 is used.

Further, the urine analysis device 200 according to an embodiment of the present disclosure can adjust individual test times and produce an accurate test result through scanning tailored to each test item.

For example, 30 seconds may be required for glucose and bilirubin, 40 seconds may be required for ketones, 45 seconds may be required for specific gravity, 60 seconds may be required for pH, protein, urobilinogen, blood and nitrite, and 2 minutes may be required for leukocytes. In this case, pH and protein can be read immediately or after more than 2 minutes, and the result for leukocytes can be confirmed based on a reaction that occurs after a delay of about 1 to 2 minutes.

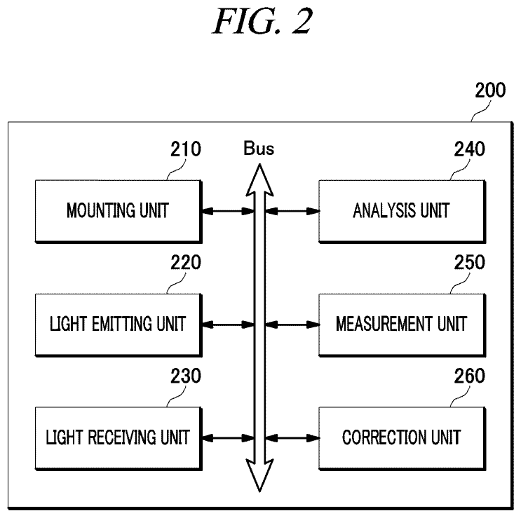

Hereafter, the urine analysis device 200 according to an embodiment of the present disclosure will be described in detail with reference to FIG. 2 to FIG. 6B.

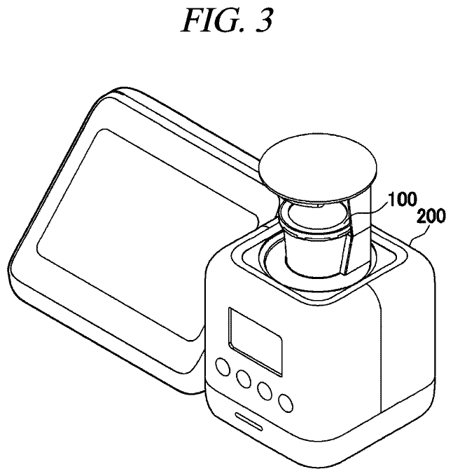

FIG. 2 is a configuration view of the urine analysis device according to an embodiment of the present disclosure, and FIG. 3 shows the urine test kit 100 mounted on the urine analysis device 200 according to an embodiment of the present disclosure.

Referring to FIG. 2, the urine analysis device 200 according to the present embodiment may include a mounting unit 210, sensor units 220 and 230, an analysis unit 240, a measurement unit 250, and a correction unit 260. However, the urine analysis device 200 illustrated in FIG. 2 is merely an embodiment of the present disclosure, and various modifications based on the components illustrated in FIG. 2 are possible. Herein, the measurement unit 250 and the correction unit 260 may be implemented by an MCU with built-in memory and peripherals. The built-in memory may store firmware and a standard colorimetric table containing reference color values for each test item.

As shown in FIG. 3, the urine test kit 100 may be mounted in the mounting unit 210. That is, the mounting unit 210 may include a predetermined space in which the urine test kit 100 can be mounted.

In this case, the mounting unit 210 may be formed to a height sufficient to cover the test paper 135 of the urine test kit 100. For example, the mounting unit 210 may have a substantially cylindrical space capable of accommodating the entire urine test kit 100. For example, the accommodating space may have a cylindrical shape concentric with the urine test kit 100 and thus can facilitate alignment between the sensor unit and the curved test strip 130.

After the urine test kit 100 is accommodated in the accommodating space, the accommodating space can be sealed with an upper cover (see FIG. 3) to suppress the introduction of external light into the accommodating space.

Furthermore, although not illustrated in the drawings, a groove or protrusion corresponding to the protrusion or groove of the container 110 may be formed in the mounting unit 210, and, thus, the urine test kit 100 can be accurately positioned in the mounting unit 210 to allow precise sensing by the sensor unit to be described below.

In this case, the sensor unit and the curved test strip 130 may be arranged as concentric circles with different radii, assuming that each of the sensor unit and the curved test strip 130 is extended with the same curvature. Accordingly, each test paper 135 of the curved test strip 130 can be aligned to face a corresponding sensor unit.

A plurality of sensor units may be provided on an inner surface of the mounting unit 210, and each sensor unit may include a pair of a light emitting unit 220 and a light receiving unit 230.

For example, the sensor units may be arranged on at least a part of an inner wall of the mounting unit 210 and oriented toward the center of the accommodating space. Thus, when the urine test kit 100 is mounted in the mounting unit 210, the sensor units are aligned to face the curved test strip 130 and can scan the test papers 135.

In an embodiment, the sensor units are formed on a flexible printed circuit board (FPCB) and can be easily positioned on the inner wall of the mounting unit 210. That is, the FPCB may be disposed on at least a part of an inner wall in the cylindrical accommodating space of the mounting unit 210, and the sensor units may be formed on the FPCB disposed on the inner wall in the cylindrical accommodating space.

The light emitting unit 220 may emit light onto the test paper 135 of the test strip 130 provided in the urine test kit 100, and may be configured as, for example, a white light LED sensor. For example, the light emitting unit 220 may be composed of a high-brightness white LED driven by a constant current control method and a MOSFET capable of changing a current applied to the LED in response to a PWM control signal from the MCU.

In an embodiment, the LED may be a high-brightness white LED with a color temperature of 5600 K.

As described above, the sensor units are aligned to face the curved test strip 130, and, thus, a plurality of light emitting units 220 is positioned to correspond to the test papers 135 included in the curved test strip 130 and emits light onto the test papers 135. Thus, it is possible to provide sufficient illuminance to the test papers 135.

Further, the light receiving unit 230 may receive light reflected from the test papers 135. For example, the light receiving unit 230 may be configured as a light receiving sensor with a 16-bit resolution.

The light receiving unit 230 may be an image sensor configured to detect the color of the test paper 135 of the curved test strip 130 based on the light reflected from the test paper 135. For example, the light receiving unit 230 may be an RGB color sensor capable of detecting red, green and blue light, or an RGBW color sensor, such as VEML6040, capable of detecting red, green, blue and white light, using an I²C interface.

The VEML6040 may be a component having L×W×H of 2.0 mm×1.25 mm×1.0 mm, with each channel (R, G, B, W) having a 16-bit resolution.

Furthermore, the light receiving unit 230 and the MCU may be connected by an I²C bus, which allows the mater MCU to read RGBW data detected by the slave light receiving unit 230.

The light emitting unit 220 and the light receiving unit 230 may be appropriately arranged or separated to suppress the direct introduction of light into the light receiving unit 230. Therefore, each light receiving unit 230 may be configured to receive only the reflected light of the light emitted from the corresponding light emitting unit 220.

Meanwhile, the urine analysis device 200 of the present embodiment may further include a substrate unit 201 that supports the sensor units 220 and 230. That is, the substrate unit 201 may be provided on the inner surface of the mounting unit 210, and a plurality of sensor units 220 and 230 may be arranged on the substrate unit 201 at predetermined intervals.

In the present embodiment, the substrate unit 201 may be configured as an FPCB to be arranged on the inner wall of the mounting unit 210 with the same or similar curvature as the test strip 130, which is bent along the circumference of the container 110. The substrate unit 201 may be configured to read various types of samples and formed with sufficient flexibility and thinness to maintain the pliability of the material.

After the urine test kit 100 is mounted in the mounting unit 210, the analysis unit 240 according to an embodiment of the present disclosure may identify the type of urine test kit 100 (11-point kit, 7-point kit, or 4-point kit) based on the number of test items.

The correction unit 260 may select available sensor units from among the plurality of sensor units 220 and 230 based on data received from the plurality of sensor units 220 and 230.

In an embodiment, data related to optical signals received by the plurality of sensor units (e.g., the light receiving units 230) may be transmitted to the correction unit 260. The correction unit 260 may select available sensor units 220 and 230 positioned to best detect the test paper 135 of the urine test kit 100 from among the plurality of densely arranged sensor units 220 and 230.

Table 2 below shows matching data including available sensor units with color sensors for each test paper 135 when the urine test kit 100 (11-point kit) includes 11 test papers 135 in the test strip 130.

TABLE 2

| No. | Test Item | Test Paper | Available Sensor Unit |
|---|---|---|---|
| 1 | UT1 | TP1 | Light Emitting Unit 1, |
| | | | Light Receiving Unit 1 |
| 2 | UT2 | TP2 | Light Emitting Unit 2, |
| | | | Light Receiving Unit 2 |
| 3 | UT3 | TP3 | Light Emitting Unit 3, |
| | | | Light Receiving Unit 3 |
| 4 | UT4 | TP4 | Light Emitting Unit 4, |
| | | | Light Receiving Unit 4 |
| 5 | UT5 | TP5 | Light Emitting Unit 5, |
| | | | Light Receiving Unit 5 |
| 6 | UT6 | TP6 | Light Emitting Unit 6, |
| | | | Light Receiving Unit 6 |
| 7 | UT7 | TP7 | Light Emitting Unit 7, |
| | | | Light Receiving Unit 7 |
| 8 | UT8 | TP8 | Light Emitting Unit 8, |
| | | | Light Receiving Unit 8 |
| 9 | UT9 | TP9 | Light Emitting Unit 9, |
| | | | Light Receiving Unit 9 |
| 10 | UT10 | TP10 | Light Emitting Unit 10, |
| | | | Light Receiving Unit 10 |
| 11 | UT11 | TP11 | Light Emitting Unit 11, |
| | | | Light Receiving Unit 11 |

In the present embodiment, the analysis unit 240 analyzes urine based on data received from the available sensor units. Specifically, the urine can be analyzed based on color information received from the sensor units 220 and 230, and as described above, the test time can be set according to each test item.

When the urine test kit 100 is mounted in the mounting unit 210, the analysis unit 240 sequentially detects the colors of the test papers sensed by the available sensor units based on the data of the available sensor units. Then, the analysis unit 240 compares the detected colors with reference colors for the corresponding test items in the standard colorimetric table, determines the levels of the test items, and derives the test result by analyzing the test items and information of the examinee.

Hereafter, the test strip 130 including, for example, less than 11 test papers 135 will be described.

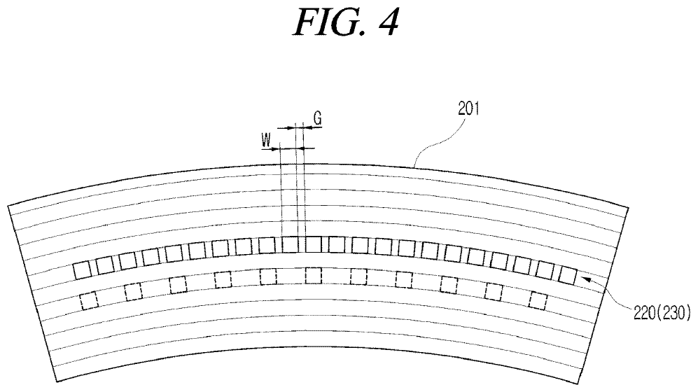

FIG. 4 is a planar figure of the substrate unit 201 of the urine analysis device 200 according to an embodiment of the present disclosure.

In the urine analysis device 200 according to an embodiment of the present disclosure, the number of sensor units 220 and 230 may be set greater than the number of test papers 135 of the test strip 130 in the urine test kit 100.

Specifically, as shown in FIG. 4, the sensor units 220 and 230 may be arranged at predetermined intervals along one direction of the substrate unit 201, for example, a circumferential direction of the mounting unit 210 or the container 110. For example, the number of test papers 135 in the test strip 130 used in the urine analysis device 200 of the present embodiment may be 11, 7, or 4, and the number of sensor units 220 and 230 may be 22 (11 light emitting units and 11 light receiving units).

Also, as shown in FIG. 4, the plurality of sensor units 220 and 230 may be densely arranged such that a gap G between the sensor units 220 and 230 is smaller than a width W of each individual sensor unit in one direction. Further, the density of the sensor units 220 and 230 arranged in the space may be set higher than that of the test papers 135.

Since the sensor units 220 and 230, which are greater in number than the test papers 135, are densely arranged as described above, it is possible to analyze various urine test kits 100 with different positions or numbers of test papers 135. Therefore, without the need for separate devices for different urine test kits 100 varying in the types or numbers of test items and the sizes of containers 110, the urine analysis device 200 according to the present embodiment can effectively perform analysis.

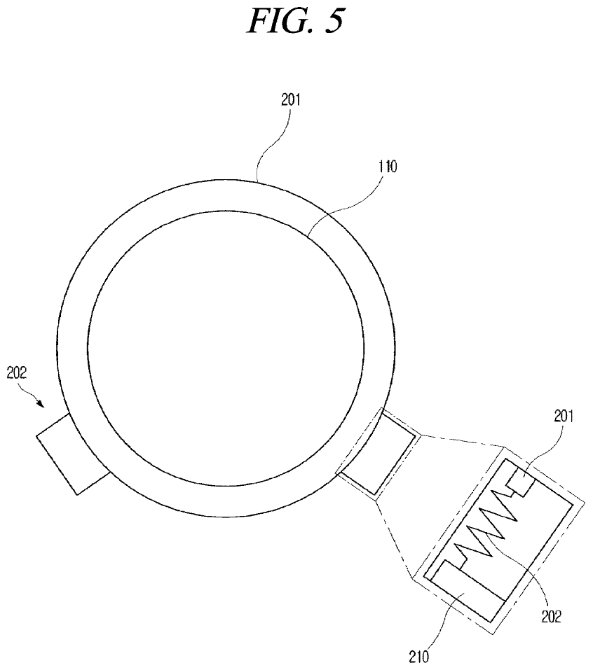

FIG. 5 illustrates the state in which the substrate unit 201 shown in FIG. 4 is disposed to surround the urine test kit 100.

In an embodiment of the present disclosure, the substrate unit 201 may be formed to allow deformation. The substrate unit 201, which may be formed of a ductile material, may include notches between the sensor units 220 and 230 to allow stretching or deformation in one direction. As the substrate unit 201 is stretched or deformed, a gap between the plurality of sensor units 220 and 230 may vary.

To enable stretching of the substrate unit 201, the urine analysis device 200 of the present embodiment may further include a connection unit 202. As shown in FIG. 5, both ends of the connection unit 202 may be fixed respectively to the mounting unit 210 and the substrate unit 201. Also, the connection unit 202 may be elastically deformed in length by user manipulation or applied force and thus can pull or push the substrate unit 201 in one direction.

For example, when the substrate unit 201 is pulled, it may elongate within an allowable range, which increases the gap between the plurality of sensor units 220 and 230. When the pulling force is released or a pushing force is applied toward the substrate unit 201, the gap between the plurality of sensor units 220 and 230 may be decreased. Further, by pulling or pushing the substrate unit 201, the substrate can be brought into close contact with the container 110 so that the distance between the sensor units 220 and 230 and the test papers 135 is suitable for analysis.

In the present embodiment, by arranging the plurality of sensor units 220 and 230 at a high density and varying the gap between the sensor units 220 and 230 in the urine analysis device 200, it is possible to analyze various urine test kits 100 with different sizes or curvature radii of the container 110 and different numbers or positions of test papers 135. Furthermore, the analysis can still be performed even if there is a variation in the mounting position of the urine test kit 100 in the mounting unit 210.

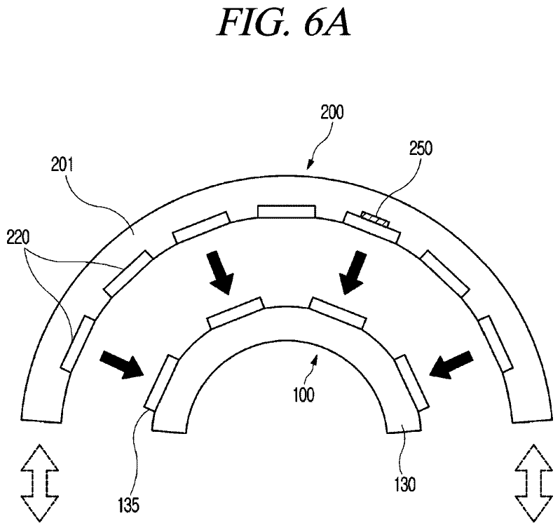

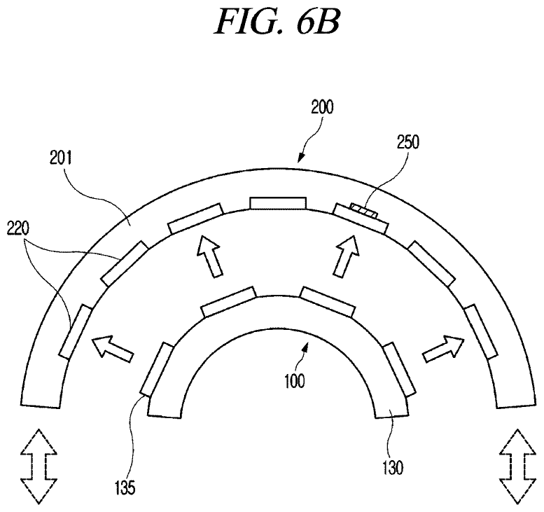

FIG. 6A and FIG. 6B are diagrams for explaining methods of acquiring color information of each of the plurality of test papers 135 by emitting light from the urine analysis device 200 according to an embodiment of the present disclosure.

In FIG. 6A and FIG. 6B, the sensor unit is illustrated as having a configuration in the light emitting unit 220 and the light receiving unit 230 are integrally formed, but is not limited thereto. The light emitting sensor and the light receiving sensor may also be separately formed.

As shown in the drawings, some of the plurality of sensor units 220 and 230, which is densely arranged relative to the test papers 135, may be positioned to correspond to at least one of the test papers 135 included in the curved test strip 130.

As shown in FIG. 6A, the light emitting unit 220 may emit light toward the corresponding test strip 130. Further, as shown in FIG. 6B, the light receiving unit 230 may receive light reflected from the corresponding test paper 135.

In the present embodiment, even if all the light emitting units 220 emit light toward the test strip 130, the light receiving unit located at a position corresponding to the test paper 135 receives light effective for analysis. Thus, the sensor unit with the light receiving unit positioned to correspond to the test paper 135 can serve as an available sensor unit. FIG. 6A and FIG. 6B illustrate light emission and reception in the available sensor unit. However, before the available sensor unit is selected, all the light emitting units 220 and light receiving units 230 may operate simultaneously.

Table 3 below shows matching data including available sensor units with color sensors for each test paper 135 when the urine test kit 100 (7-point kit) includes 7 test papers 135 in the test strip 130.

TABLE 3

| | Available/ | Color | | |
|---|---|---|---|---|
| Sensor Unit | Unavailable | Value | Test Pad | Test Item |
| Light Emitting Unit 1, | Available | a | TP1 | UT1 |
| Light Receiving Unit 1 | | | | |
| Light Emitting Unit 2, | Unavailable | — | — | — |
| Light Receiving Unit 2 | | | | |
| Light Emitting Unit 3, | Available | b | TP2 | UT2 |
| Light Receiving Unit 3 | | | | |
| Light Emitting Unit 4, | Unavailable | — | — | — |
| Light Receiving Unit 4 | | | | |
| Light Emitting Unit 5, | Available | c | TP3 | UT3 |
| Light Receiving Unit 5 | | | | |
| Light Emitting Unit 6, | Available | d | TP4 | UT4 |
| Light Receiving Unit 6 | | | | |
| Light Emitting Unit 7, | Available | e | TP5 | UT5 |
| Light Receiving Unit 7 | | | | |
| Light Emitting Unit 8, | Unavailable | — | — | — |
| Light Receiving Unit 8 | | | | |
| Light Emitting Unit 9, | Available | f | TP6 | UT6 |
| Light Receiving Unit 9 | | | | |
| Light Emitting Unit 10, | Unavailable | — | — | — |
| Light Receiving Unit 10 | | | | |
| Light Emitting Unit 11, | Available | g | TP7 | UT7 |
| Light Receiving Unit 11 | | | | |

Table 4 below shows matching data including available sensor units with color sensors for each test paper 135 when the urine test kit 100 (4-point kit) includes 4 test papers 135 in the test strip 130.

TABLE 4

| | Available/ | Color | | |
|---|---|---|---|---|
| Sensor Unit | Unavailable | Value | Test Pad | Test Item |
| Light Emitting Unit 1, | Unavailable | — | — | — |
| Light Receiving Unit 1 | | | | |
| Light Emitting Unit 2, | Available | a | TP1 | UT1 |
| Light Receiving Unit 2 | | | | |
| Light Emitting Unit 3, | Unavailable | — | — | — |
| Light Receiving Unit 3 | | | | |
| Light Emitting Unit 4, | Unavailable | — | — | — |
| Light Receiving Unit 4 | | | | |
| Light Emitting Unit 5, | Available | b | TP2 | UT2 |
| Light Receiving Unit 5 | | | | |
| Light Emitting Unit 6, | Unavailable | — | — | — |
| Light Receiving Unit 6 | | | | |
| Light Emitting Unit 7, | Unavailable | — | — | — |
| Light Receiving Unit 7 | | | | |
| Light Emitting Unit 8, | Available | c | TP3 | UT3 |
| Light Receiving Unit 8 | | | | |
| Light Emitting Unit 9, | Unavailable | — | — | — |
| Light Receiving Unit 9 | | | | |
| Light Emitting Unit 10, | Unavailable | — | — | — |
| Light Receiving Unit 10 | | | | |
| Light Emitting Unit 11, | Available | d | TP4 | UT4 |
| Light Receiving Unit 11 | | | | |

According to an embodiment of the present disclosure, even if the urine test kit 100 varies in type (11-point kit, 7-point kit, or 4-point kit), urine analysis can be performed just by mounting the urine test kit 100 in the urine analysis device 200 without the need for a separate matching process between the color sensors and the test papers.

Therefore, the urine analysis device 200 of the present disclosure provides excellent compatibility since it is possible to relatively freely design of the number and size of test papers 135 in the urine test kit 100.

Meanwhile, in the urine analysis device 200 according to an embodiment of the present disclosure, a color value of a sample to be analyzed may be affected and changed due to changes in temperature or humidity of the sensor units 220 and 230 and its surroundings. The urine analysis device 200 according to an embodiment of the present disclosure can enhance the accuracy of analysis by measuring temperature or humidity through the measurement unit 250 and either maintaining the value within a predetermined range or compensating for any variations.

Specifically, the measurement unit 250 may measure the temperature of the mounting unit 210, the sensor units 220 and 230, the substrate unit 201, or its surroundings via a temperature sensor (not shown). Further, the correction unit 260 may correct a duty ratio of light emitted by the sensor units 220 and 230 based on the temperature measured by the measurement unit 250.

That is, when the light emitting unit 220 continuously emits light and increases in temperature, the correction unit 260 may increase a duty ratio of the light emitting unit 220 via PWM control. For example, by increasing the duty ratio by 2% for every 2° C. increase in temperature, the output of the light emitting unit 220 can be increased at higher temperatures and deviations in optical data caused by temperature changes can be compensated.

The measurement unit 250 may detect whether external light enters the accommodating space of the mounting unit 210 while the urine test kit 100 is mounted, using an illuminance sensor (not shown) provided within the accommodating space. When the external light is detected entering the accommodating space, an alarm can be generated via a speaker (not shown), display, or the like.

Also, the measurement unit 250 may measure the humidity of the mounting unit 210, the sensor units 220 and 230, the substrate 201, or its surroundings. The correction unit 260 may compare the measured humidity measured by the measurement unit 250 with a predetermined value or range. When the humidity is not equal to a desirable value or within a desirable range, the urine analysis device 200 can generate an alarm, which prompts the user to take corrective action, such as ventilation, to bring the humidity within the desirable range.

Further, the correction unit 260 of the urine analysis device 200 according to the present embodiment may calculate the distance between the sensor units 220 and 230 and the test papers 135. In the urine analysis device 200 of the present embodiment, the container 110 may be inaccurately placed in the mounting unit 210 depending on user manipulation. In response, the correction unit 260 may calculate the distance between the sensor units 220 and 230 and the test papers 135 based on the data received from the sensor units 220 and 230. The correction unit 260 may compare the calculated distance with a predetermined value or range. When the distance is not equal to a desirable value or within a desirable range, the urine analysis device 200 of the present embodiment can generate an alarm, which prompts the user to take corrective action. For example, the user may be guided to place the urine test kit 100 in the mounting unit 210 again.

Meanwhile, the urine analysis device 200 according to an embodiment of the present disclosure may further include an output unit. For example, the urine analysis device 200 may be equipped with a display to present to present the analysis result of the tested urine. The output unit can also output the above-described alarm to the user via visual or auditory information. Further, the output unit can transmit the urine analysis result and other data to a device through a communication means.

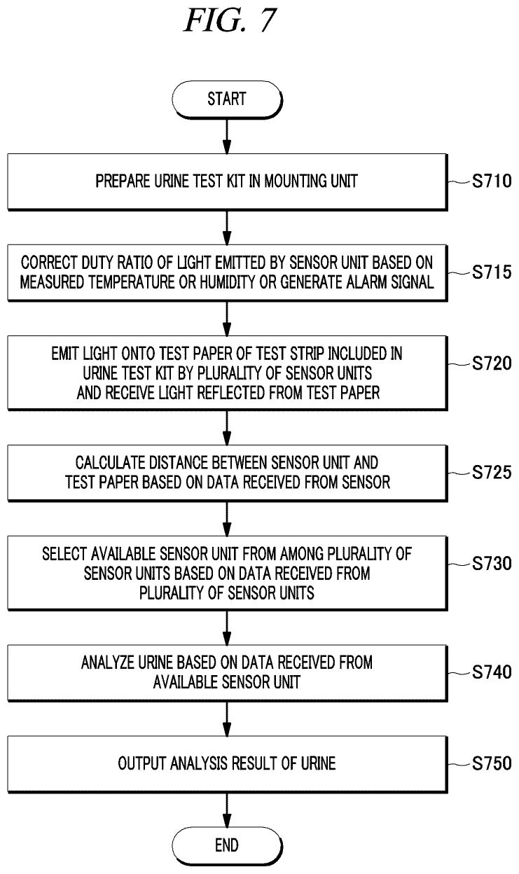

FIG. 7 is a flowchart of a urine analysis method using the urine test kit 100 according to an embodiment of the present disclosure. The urine analysis method performed by the urine analysis device 200 illustrated in FIG. 7 includes the processes time-sequentially performed by the urine analysis device 200 illustrated in FIG. 1 to FIG. 6B. Therefore, the descriptions of the processes may also be applied to the urine analysis method performed by the urine analysis device 200 according to the embodiment illustrated in FIG. 1 to FIG. 6B even though they are omitted hereinafter.

In a process S710, a urine test kit may be prepared. The urine test kit 100 in which a patient's urine has been collected may be positioned in the mounting unit 210 of the urine analysis device 200.

In a process S720, a plurality of sensor units may emit light onto test papers of a test strip included in the urine test kit and receive light reflected from the test papers. The plurality of sensor units 220 and 230 may emit and receive light either simultaneously or sequentially.

In a process S730, available sensor units are selected from among the plurality of sensor units. Based on the data received from the plurality of sensor units 220 and 230, sensor units that receive valid data suitable for urine analysis may be selected as available sensor units.

In a process S740, urine is analyzed based on data received from the available sensor units. The components of urine may be analyzed based on color information in the received data.

In a process S750, the analysis result of the urine may be output. The analysis result may be output to the user via an output unit such as a display.

Meanwhile, in an embodiment of the present disclosure, the urine test kit 100 may include a reference kit to select available sensor units. The reference kit may be a urine test kit which has a specific number and arrangement of test papers and in which urine has not been collected.

In the process S730 of selecting available sensor units, the data received from the plurality of sensor units 220 and 230 regarding the test papers 135 of the test strip 130 included in the reference kit may be used as a criterion for selecting available sensor units. The reference kit may be used to select available sensor units by comparing the color information received from the sensor units or comparing color information depending on the positions of a substrate unit and test papers.

As described above, the urine analysis device according to an embodiment of the present disclosure includes sensor units, which are greater in number than the test papers in the urine test kit, and, thus. Therefore, sensor units at certain positions in one direction (for example, odd-numbered sensor units, see FIG. 6A and FIG. 6B) can be selected as available sensor units. Alternatively, sensor units up to a certain position (for example, up to the 11th sensor unit) may be selected as available sensor units.

The urine analysis method of the present embodiment may further include a process of correcting a duty ratio of light emitted by the sensor unit based on the temperature of the sensor unit or its surroundings (S715). In this case, the temperature may be a value measured by the above-described measurement unit 250.

The urine analysis method of the present embodiment may also include a process of generating an alarm signal when the measured humidity of the sensor unit or its surroundings does not satisfy a predetermined desirable value or range by comparing it with the predetermined value or range (S715). The humidity may be measured by the above-described measurement unit 250, and the alarm output by the output unit may prompt the user to take appropriate action.

The urine analysis method of the present embodiment may further include a process of calculating the distance between the sensor unit and the test paper based on the data received from the sensor unit (S725). The calculated distance may be compared with a predetermined value or range, and an alarm signal may be generated according to predetermined conditions to prompt the user to take appropriate action. By prompting the user to perform manipulation, it is possible to suppress the reception of light or color information that is unsuitable for urine analysis.

In the descriptions above, the processes S710 to S750 may be divided into additional processes or combined into fewer processes depending on an embodiment. In addition, some of the processes may be omitted and the sequence of the processes may be changed if necessary.

The urine analysis method illustrated in FIG. 7 can be implemented as a computer program stored in a medium to be executed by a computer or a storage medium including instructions executable by a computer. Also, the urine analysis method illustrated in FIG. 7 can be implemented as a computer program stored in a medium to be executed by a computer.

A computer-readable medium can be any usable medium which can be accessed by the computer and includes all volatile/non-volatile and removable/non-removable media. Further, the computer-readable medium may include all computer storage media. The computer storage media include all volatile/non-volatile and removable/non-removable media embodied by a certain method or technology for storing information such as computer-readable instruction code, a data structure, a program module or other data.

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

We claim:

1. A urine analysis device, comprising:
a mounting unit in which a test kit is mounted, the test kit including a container into which urine is collected and a curved test strip which is attached along an inner or outer circumferential surface of the container and includes a plurality of test papers provided to test the collected urine;
a plurality of sensor units, each composed of a pair of a light emitting unit configured to emit light onto each of the plurality of test papers of the test kit mounted on the mounting unit and a light receiving unit configured to detect a color of each test paper based on the light reflected from each of the plurality of test papers; and
an analysis unit that analyzes the urine based on the color information of the test papers detected by the light receiving units.

2. The urine analysis device of claim 1,
wherein each of the sensor units performs scanning tailored to test items associated with its corresponding test paper.

3. The urine analysis device of claim 1, further comprising:
a correction unit configured to measure a temperature of the mounting unit or the plurality of sensor units via a temperature sensor and correct a duty ratio of light emitted by the light emitting unit based on the measured temperature.

4. The urine analysis device of claim 1,
wherein a protrusion or groove configured to guide the container to a mounting position is formed at the bottom of the container when the mounting unit is mounted, and
a groove or protrusion corresponding to the protrusion or groove of the container is formed in the mounting unit.

5. The urine analysis device of claim 1,
wherein the number of the plurality of sensor units is different from the number of the plurality of test papers.

6. The urine analysis device of claim 1,
wherein the number of the plurality of sensor units is equal to the number of the plurality of test papers.

7. The urine analysis device of claim 1,
wherein the mounting unit is formed to a height sufficient to cover the plurality of test papers.

8. The urine analysis device of claim 1,
wherein the mounting unit is formed to a height sufficient to accommodate the entire urine test kit.

9. The urine analysis device of claim 8,
wherein after the urine test kit is mounted in the mounting unit, an accommodating space of the mounting unit is sealed with an upper cover.

10. The urine analysis device of claim 1,
wherein an accommodating space of the mounting unit has a cylindrical shape concentric with the urine test kit.

11. The urine analysis device of claim 10,
wherein the plurality of sensor units is formed on a substrate provided on at least a part of an inner wall in the cylindrical accommodating space and formed of a ductile material.

12. The urine analysis device of claim 1,
wherein the plurality of sensor units is formed on a substrate provided on an inner wall of the mounting unit with the same curvature as the curved test strip, which is bent along a circumference of the container, and formed of a ductile material.

13. The urine analysis device of claim 1,
wherein the plurality of sensor units is aligned to face the test papers of the curved test strip.

14. The urine analysis device of claim 1,
wherein the plurality of sensor units is arranged on at least a part of an inner wall of the mounting unit and oriented toward a center of an accommodating space of the mounting unit.

15. A urine analysis device, comprising:
a mounting unit in which a test kit is mounted, the test kit including a container into which urine is collected and a curved test strip which is attached along an inner or outer circumferential surface of the container and includes a plurality of test papers provided to test the collected urine;
a substrate unit provided on an inner surface of the mounting unit and formed of a ductile material;
a sensor unit provided on the substrate unit and composed of a light emitting unit configured to emit light to the plurality of test papers and a light receiving unit configured to detect a color of the plurality of test papers based on the light reflected from the plurality of test papers; and
an analysis unit that analyzes the urine based on the color information of the test papers detected by the light receiving unit.

16. The urine analysis device of claim 15,
wherein the substrate unit formed of the ductile material is provide on an inner wall of the mounting unit with the same curvature as the curved test strip, which is bent along a circumference of the container.

17. The urine analysis device of claim 15,
wherein an accommodating space of the mounting unit has a cylindrical shape concentric with the urine test kit.

18. The urine analysis device of claim 17,
wherein the substrate unit formed of the ductile material is provided on at least a part of an inner wall in the cylindrical accommodating space.
